(19) United States
(12) Patent Application Publication
(10) Pub. No.: US 2025/0032550 A1
(43) Pub. Date: Jan. 30, 2025

(54) A NOVEL WAY FOR BRAIN-SPECIFIC DELIVERY OF MOLECULES FOR THE TREATMENT OF BRAIN DISEASES

(71) Applicant: West Virginia University Board of Governors on behalf of West Virginia University, Morgantown, WV (US)

(72) Inventors: Saumyendra SARKAR, Morgantown, WV (US); James W. SIMPKINS, Morgantown, WV (US)

(73) Assignee: West Virginia University Board of Governors on behalf of West Virginia University, Morgantown, WV (US)

(21) Appl. No.: 18/710,756

(22) PCT Filed: Nov. 15, 2022
(86) PCT No.: PCT/US2022/079854

§ 371 Date: May 16, 2024

Related U.S. Application Data
(60) Provisional application No. 63/264,123, filed on Nov. 16, 2021.

(51) Int. Cl. A61K 35/28; A61K 47/64; A61K 47/69; C07K 14/00; C12N 5/0775; C12N 15/85

(52) U.S. Cl. CPC A61K 35/28; A61K 47/64; A61K 47/6901; C07K 14/00; C12N 5/0663; C12N 15/85; C12N 2800/107

(57) ABSTRACT

A composition comprising a brain targeted exosome derived from mesenchymal stem cells for systemically delivering an agent to target cells or tissues in the central nervous system of a patient, wherein the exosome expresses blood brain barrier (BBB) crossing central nervous system (CNS) specific AAV synthetic capsid (CAP) peptide. A method is provided for genetically engineering exosomes comprising joining a selected functional AAV capsid domain specific peptide (CAP), and fusing said CAP to lysosome-associated membrane glycoprotein (Lanp2b) to form a CAP-Lam2b fusion protein, and expressing CAP-lamp2b fusion protein on the surface of a mesenchymal stem cell derived exosome.

### CROSS-REFERENCE TO RELATED APPLICATION

This utility non-provisional patent application claims the benefit of priority to International Application No. PCT/US22/79854, filed on Nov. 15, 2022, which claims priority to U.S. Provisional Patent Application Ser. No. 63/264,123, filed Nov. 16, 2021. The entire contents of International Application No. PCT/US22/79854 and U.S. Provisional Patent Application Ser. No. 63/264,123 are incorporated by reference into this utility non-provisional patent application as if fully written herein.

### STATEMENT REGARDING FEDERALLY SPONSORED RESEARCH OR DEVELOPMENT

Not applicable.

### SEQUENCE LISTING

A SEQUENCE LISTING having SEQ ID NO:1 and SEQ ID NO:2 in electronic computer-readable form named “0074539-000153.xml” which was created on May 15, 2024 and is 3,118 bytes in size accompanies this application and is electronically submitted herewith via EFS-Web, and such SEQUENCE LISTING is incorporated into this application by reference in its entirety as if fully written herein.

### BACKGROUND OF THE INVENTION

### 1. Field of the Invention

This invention provides an allogenic or autologous mesenchymal stem cell derived exosomes as a systemic drug delivery vehicle that are non-immunogenic. This invention provides a genetic engineering of exosomes for improving its ability to transduce brain cells and not transduce cells in the liver and other off-target organs.

### 2. Background Art

The blood brain barrier (BBB) in the central nervous system prevents the brain uptake of circulating small molecule drugs, protein therapeutics, RNAi drugs, and other therapeutic genes. The only way the drug or gene can be distributed widely in the brain is the transvascular route following injection into the blood stream. However, this transvascular route requires the ability to undergo transport across the BBB Adena-associated viruses (AAVs) have been established as safe, well tolerated viral vectors for gene delivery, and some AAV-serotypes can target brain along with other organs after intravenous injection. Very recently attempts were made to engineer AAV capsids for efficient noninvasive gene delivery to the central and peripheral nervous systems. For the AAV vector gene transfer technology, several hurdles have emerged in both preclinical studies and clinical trials and the barriers remain concerning the restriction in size of the gene need to be packaged, reducing the production of neutralizing antibodies, activation of capsid-specific CDS+T cells, and targeting specific organ/cell-type.

Brain targeted exosomes from mesenchymal stem cell (MSC) can be created by engineering the exosomes to express CNS specific rabies virus glycoprotein (RVG) peptide and it has been reported that intravenous injection of these engineered exosomes delivered siRNA specifically to the brain. However, RVG-exosomes also transduce cells in the liver and other off-target organs. The liver is an immunologically active organ, with large populations of phagocytic cells that play a critical role in immune activation, making avoiding the liver particularly important for avoiding strong systemic immune responses.

### SUMMARY OF THE INVENTION

The present invention is intended to solve many of the above problems by first choosing the allogenic or autologous mesenchymal stem cell derived exosomes as a systemic drug delivery vehicle that are non-immunogenic and secondly genetic engineering of exosomes for improving its ability to transduce brain cells and not transduce cells in liver and other off-target organs.

In certain embodiments of this invention, a composition for systemically delivering agents to target cells or tissues in the central nervous system of a patient comprising a brain targeted exosome derived from mesenchymal stem cells. In certain embodiments of this invention the composition the exosome expresses blood brain barrier (BBB) crossing central nervous system (CNS) specific AAV synthetic capsid (CAP) peptide.

Other embodiments of this invention provide an amino acid sequence of SEQ ID NO:1 and a DNA (nucleotide) sequence of SEQ ID NO:2.

This invention provides an amino acid sequence of joined CAP and corresponding nucleotide codons cloned in the N-terminal domain of pcDNA Lamp2b-HA plasmid vector comprising:

```
5′-CAC GAC AAG GCC TAC GAC AGA CAG GCC AAG AAG AGG ggc agc ggc
        H D K A Y D R Q A K K R G S G
      Endosomal escaping signal Nuclear localization signal Spacer
GAC GGC ACC CTG GCC GTG CCC TTC AAG GCC ggc agc ggc ggc GTG AGC
  D G T L A V P F K A G S G G V S
  Insertion in variable region VIII of AAVS Spacer
ACC AAC CTG CAG AGC GGC AAC ACC CAG GCC GCC ACC ACC ggc agc GAC
  T N L Q S G N T Q A A T T G S D
Substitution in variable region IV of AAV9 Spacer
GAC GGC CAG AGC AGC AAG AGC-3′
  D G Q S S K S
```

In certain embodiments of this invention, a method for genetically engineering exosomes is provided comprising joining selected functional AAV capsid domain specific peptide (CAP), and fusing said CAP to lysosome-associated membrane glycoprotein (Lanp2b) to form CAP-Lam2b fusion protein, and expressing CAP-lamp2b fusion protein on the surface of a mesenchymal stem cell derived exosomes.

In certain embodiments of this invention, a composition is provided comprising an amino acid sequence of a joined CAP cloned in a N-terminal domain of a pcDNA Lamp2b-HA plasmid vector of SEQ ID NO:1, and optionally a pharmaceutically acceptable carrier.

### BRIEF DESCRIPTION OF THE FIGURES

The patent or application file contains at least one drawing executed in color. Copies of this patent or patent application publication with color drawings will be provided by the office upon request and payment of the necessary fee.

The figures set forth various embodiments of this invention.

FIG. 1 shows a schematic diagram for cloning capsid domain in Lamp2b expression plasmid.

FIG. 2 shows a diagnostic restriction enzyme digestion of mini plasmid DNA isolated from blunt-end ligated AMP-R transformed single bacterial colony to identify in frame cloning of CAP domain in N-terminus of Lamp2b.

FIG. 3 shows a schematic diagram for the preparation of bone marrow-mesenchymal stem cells (BM-MSCs) derived Lamp2b-capexosomes carrying the GFP-gene and sets forth the loading of GFP expression plasmid into the capexosomes.

FIG. 4A shows systemically delivered capexosomes crosses the blood brain barrier (BBB) and targets brain cells. FIG. 4A shows a confocal micrograph representing optical Z-sections maximum intensity projected capexosomes in a mouse brain. FIG. 4A shows DII (red) labelled exosomes that are surrounded near cell body as indicated by Dapi (blue) labelled nucleus of frontal cortical cells.

FIG. 4B shows a confocal micrograph representing optical Z-sections maximum intensity projected capexosomes in the liver of the same mouse as in FIG. 4A. FIG. 4B sets forth a liver section of the same mouse (of FIG. 4A) showing less delivery of capexosomes compared to the brain (of FIG. 4A).

FIG. 5A shows systemically delivered GFP-capexosomes crosses the BBB and targets frontal cortical brain cells. FIG. 5A shows confocal micrographs representing optical Z-sections maximum intensity projected capexosomes in a mouse brain. FIG. 5A shows DII (red) labelled exosomes n the frontal cortex region of the mouse brain.

FIG. 5B shows systemically delivered GFP-capexosomes crosses the BBB and targets frontal cortical brain cells. FIG. 5B shows confocal micrographs representing optical Z-sections maximum intensity projected capexosomes in a mouse brain. FIG. 5B shows GFP (green) expression in the same frontal cortical mouse brain cells of FIG. 5A.

FIG. 5C shows systemically delivered GFP-capexosomes crosses the BBB and targets frontal cortical brain cells. FIG. 5C shows confocal micrographs representing optical Z-sections maximum intensity projected capexosomes in a mouse brain. FIG. 5C shows the same frontal cortical region of FIG. 5A merged Blue+Red.

FIG. 5D shows systemically delivered GFP-capexosomes crosses the BBB and targets hippocampus region of the mouse brain cells. FIG. 5D shows confocal micrographs representing optical Z-sections maximum intensity projected capexosomes in a mouse brain. FIG. 5D shows DII (red) labelled exosomes in the hippocampus region of the mouse brain.

FIG. 5E shows systemically delivered GFP-capexosomes crosses the BBB and targets hippocampus region of the mouse brain cells. FIG. 5E shows confocal micrographs representing optical Z-sections maximum intensity projected capexosomes in a mouse brain. FIG. 5E shows GFP (green) expression in the same hippocampus mouse brain cells of FIG. 5D.

FIG. 5F shows systemically delivered GFP-capexosomes crosses the BBB and targets hippocampus region of the mouse brain cells. FIG. 5F shows confocal micrographs representing optical Z-sections maximum intensity projected capexosomes in a mouse brain. FIG. 5E shows the same hippocampus region of FIG. 5D merged Blue+Red.

FIG. 6A shows systemically delivered GFP-capexosomes crosses the BBB and targets hippocampus cells of the mouse brain. FIG. 6A shows a tile scan stitched confocal micrograph representing optical Z-sections maximum intensity projected capexosomes in the hippocampus of a mouse brain. FIG. 6A shows the NeuN antibody (white) labelled hippocampal neurons.

FIG. 6B shows systemically delivered GFP-capexosomes crosses the BBB and targets hippocampus cells of the mouse brain. FIG. 6B shows a tile scan stitched confocal micrograph representing optical Z-sections maximum intensity projected capexosomes in the hippocampus of a mouse brain. FIG. 6B shows DH-labelled (red) capexosomes.

FIG. 6C shows systemically delivered GFP-capexosomes crosses the BBB and targets hippocampus cells of the mouse brain. FIG. 6C shows a tile scan stitched confocal micrograph representing optical Z-sections maximum intensity projected capexosomes in the hippocampus of a mouse brain. FIG. 6C shows a GFP (green) expression in the same hippocampal area as FIG. 6A.

### DETAILED DESCRIPTION OF THE INVENTION

The invention disclosed here provides methods and compositions useful for systemic delivering agents to target cells or tissues for example brain cells in the central nervous system (CNS). The composition and methods are specifically useful for delivering agents across the blood-brain barrier (BBB). The present invention also provides methods of using the composition provided by the present invention to deliver therapeutic agents for the treatment of neurologically related disorders, for example Alzheimer's disease.

As used herein, the term “patient” means members of the animal kingdom, including but not limited to, human beings.

As used herein, the term “effective amount” or “therapeutically effective amount” refers to that amount of any of the present compounds, salts thereof, and/or compositions required to bring about a desired effect in a patient. The desired effect will vary depending upon the illness or disease state being treated. For example, the desired effect may be treating brain specific diseases such as for example Alzheimers disease. On its most basic level, a therapeutically effective amount is that amount of a substance needed to treat Alzheimer's disease. The term “inhibits or inhibiting” as used herein means reducing progression of disease.

As used herein, the term “therapeutically effective carrier” or “pharmaceutically acceptable carrier” refers to any pharmaceutically acceptable carrier known in the art, absent compatibility problems with the novel compounds and compositions of the invention. Generally, carriers include for example but not limited to, physiologic saline and 5% dextrose in water.

As will be understood by one skilled in the art, a therapeutically effective amount of said compound can be administered by any means known in the art, including but not limited to, injection, parenterally, intravenously, intraperitoneally, orally or, where appropriate, topically.

It is well within the skill of one practicing in the art to determine what dosage, and the frequency of this dosage, which will constitute a therapeutically effective amount for each individual patient, depending on the severity or progression of the central nervous system and brain specific disease. It is also within the skill of one practicing in the art to select the most appropriate method of administering the compounds and compositions based upon the needs of each patient.

In order to achieve brain cell specific targeting, the present invention provides a method for genetically engineering exosomes by joining various selected functional AAV capsid domain specific peptide (we call it CAP) and then fusing the CAP to lysosome-associated membrane glycoprotein (Lamp2b) followed by expressing CAP-lamp2b fusion protein on the surface of mesenchymal stem cell derived exosomes. Selection of functional AAV capsid domain specific peptide had an emphasis on AAV-particle's ability to traffic intracellularly (endosomal escaping), nuclear entry, and BBB crossing to enter brain cell.

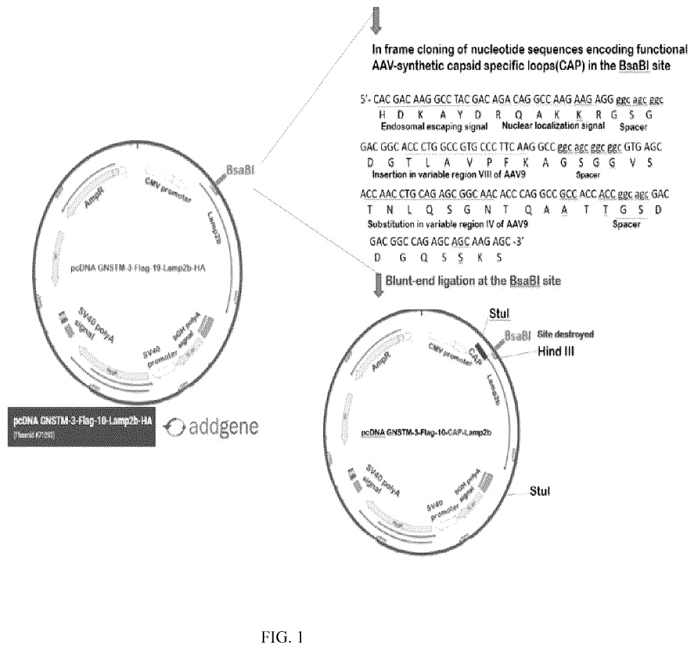

Amino acid sequences of joined CAP and their corresponding nucleotide codons used for cloning in the N-terminal domain of peDNA Lamp2b-HA plasmid vector are presented in FIG. 1. FIG. 1 shows a schematic diagram for cloning capsid domain in Lamp2b expression plasmid. Wild type AAV-capsid specific sequences used in this invention from GenBank: MP 729913.1, and from capsid protein (synthetic construct) from GenBank: ASK 12222.3.

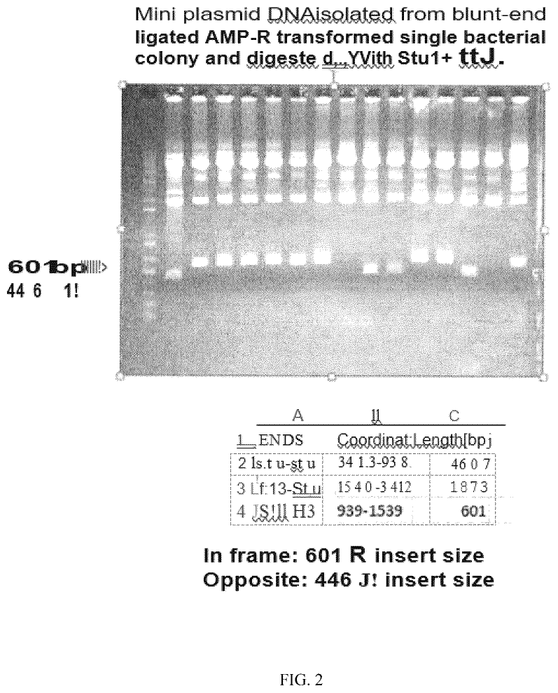

FIG. 2 shows a diagnostic restriction enzyme digestion of mini plasmid DNA isolated from blunt-end ligated AMP-R transformed single bacterial colony to identify in frame cloning of CAP domain in N-terminus of Lamp2b.

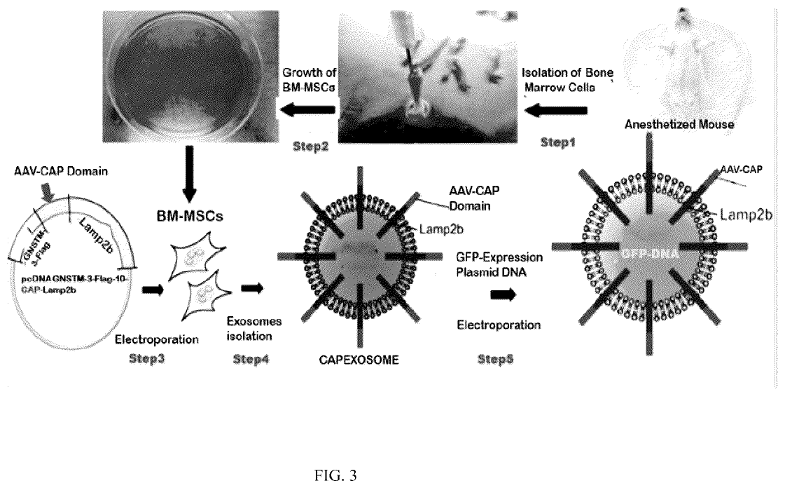

FIG. 3 shows a schematic diagram for the preparation of bone marrow-mesenchymal stem cells (BM-MSCs) derived Lamp2b-capexosomes carrying the GFP-gene and sets forth the loading of GFP expression plasmid into the capexosomes.

### Methods of this Invention

Step 1: FVB mouse bone marrow cell collection: The anesthetized FVB mice (2-3 months old) was placed in a 100-mm culture dish, and washed with 70% ethanol, Tibias and femurs were dissected; muscle, ligaments, and tendons were removed. Next, with micro dissecting scissors two ends were excised and a needle with the syringe filled with sterile phosphate buffered saline was inserted into the bone cavity and used to slowly flush the marrow out into a culture dish containing BM-MSC growth medium.

Step 2 and 3: BM-MSC colony growth WAS expanded according to the protocol described by Masoud Soleimani & Samad Nadri (A protocol for isolation and culture of mesenchymal stem cells from mouse bone marrow, NATURE PROTOCOL, VOL. 4 NO. 1 I 2009, 102-106) and used for transfection of CAP-Lamp2b expression cassette containing plasmid DNA by electroporation.

Step 4: From CAP-Lamp2b plasmid DNA transfected BM-MSC cell culture conditioned media exosomes were isolated and purified either by differential centrifugation as described in by Thery C, Amigorena S, Raposo G, Clayton A. Isolation and characterization of exosomes from cell culture supematants and biological fluids. Curr Protoc Cell Biol. 2006; Chapter 3: Unit 3.22.

Step 5: GFP expression plasmid was inserted into CAPEXOSOMES using 4-D-Nucleofector, instruments and reagents from Lonza, Basel, Switzerland, following the company's instructions.

In order to study whether capexosomes can be utilized therapeutically for the targeted delivery of gene drugs to the brain, various experiments were designed for this invention and some examples of experimental data are presented below.

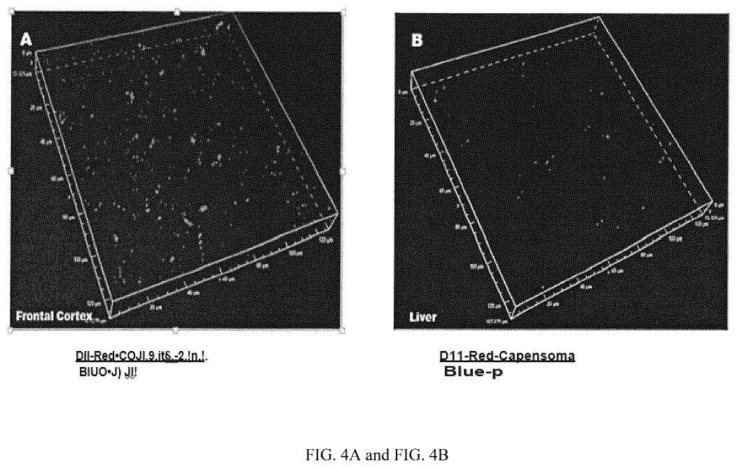

FIG. 4A shows systemically delivered capexosomes crosses the blood brain barrier (BBB) and targets brain cells. FIG. 4A shows a confocal micrograph representing optical Z-sections maximum intensity projected capexosomes in a mouse brain. FIG. 4A shows DII (red) labelled exosomes that are surrounded near cell body as indicated by Dapi (blue) labelled nucleus of frontal cortical cells.

FIG. 4B shows a confocal micrograph representing optical Z-sections maximum intensity projected capexosomes in the liver of the same mouse as in FIG. 4A. FIG. 4B sets forth a liver section of the same mouse (of FIG. 4A) showing less delivery of capexosomes compared to the brain (of FIG. 4A).

Genetically engineered mesenchymal stem cell derived exosomes (Capexosomes) were made as schematically described in the previous section. These capexosomes were then used for incorporating GFP expression plasmid DNA vector into the exosomes by elctropration using 4D-Nucleofector apparatus and using manufacturer Instructions. After electroporation exosomes are labelled with membrane staining dye DII (Red). DII stained and GFP-expression plasmid electoporated exosomes were systemically delivered by i.v. (intra-venous) injection into 4-5 months old FVB mice. The volume was 100 μl phosphate buffered saline (PBS). Capexosomes and exosomes were 5×10¹⁰ particles in 100 μl of PBS. 4 days after injection mice were perfused and brain was collected. Whole brain frozen sections (40 micron) were analyzed by confocal microscopy after counterstained with either 4,6-diamidino-2-phenylindole (DAPI) or neuronal nuclear antigen (NeuN) antibody (MAB 377, Millipore, USA) followed by Alexa 633-conjugated secondary antibody treatment.

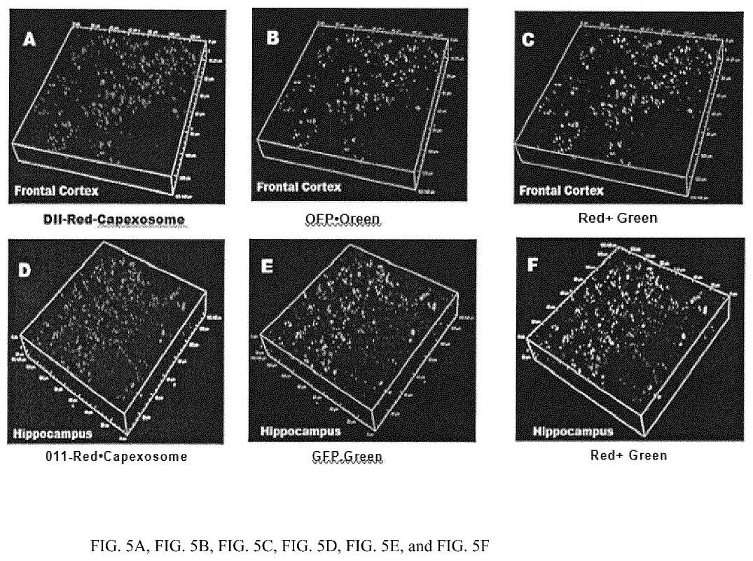

FIG. 5A shows systemically delivered GFP-capexosomes crosses the BBB and targets frontal cortical brain cells. FIG. 5A shows confocal micrographs representing optical Z-sections maximum intensity projected capexosomes in a mouse brain. FIG. 5A shows DII (red) labelled exosomes n the frontal cortex region of the mouse brain.

FIG. 5B shows systemically delivered GFP-capexosomes crosses the BBB and targets frontal cortical brain cells. FIG. 5B shows confocal micrographs representing optical Z-sections maximum intensity projected capexosomes in a mouse brain. FIG. 5B shows GFP (green) expression in the same frontal cortical mouse brain cells of FIG. 5A.

FIG. 5C shows systemically delivered GFP-capexosomes crosses the BBB and targets frontal cortical brain cells. FIG. 5C shows confocal micrographs representing optical Z-sections maximum intensity projected capexosomes in a mouse brain. FIG. 5C shows the same frontal cortical region of FIG. 5A merged Blue+Red.

FIG. 5D shows systemically delivered GFP-capexosomes crosses the BBB and targets hippocampus region of the mouse brain cells. FIG. 5D shows confocal micrographs representing optical Z-sections maximum intensity projected capexosomes in a mouse brain. FIG. 5D shows DII (red) labelled exosomes in the hippocampus region of the mouse brain.

FIG. 5E shows systemically delivered GFP-capexosomes crosses the BBB and targets hippocampus region of the mouse brain cells. FIG. 5E shows confocal micrographs representing optical Z-sections maximum intensity projected capexosomes in a mouse brain. FIG. 5E shows GFP (green) expression in the same hippocampus mouse brain cells of FIG. 5D.

FIG. 5F shows systemically delivered GFP-capexosomes crosses the BBB and targets hippocampus region of the mouse brain cells. FIG. 5F shows confocal micrographs representing optical Z-sections maximum intensity projected capexosomes in a mouse brain. FIG. 5E shows the same hippocampus region of FIG. 5D merged Blue+Red.

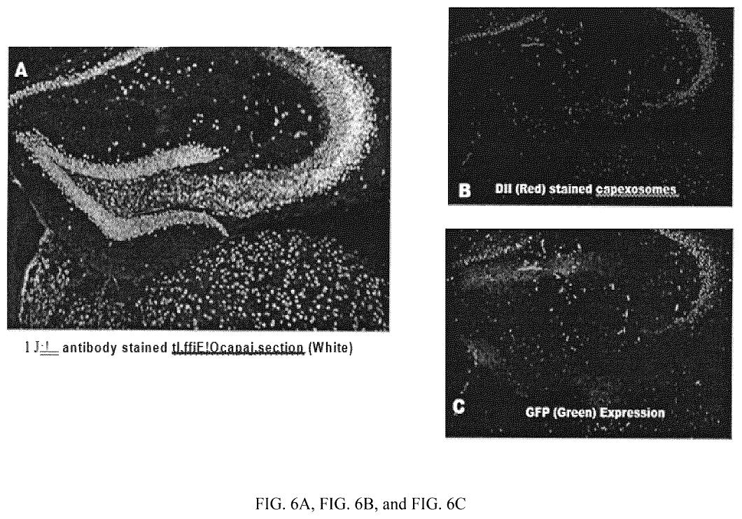

FIG. 6A shows systemically delivered GFP-capexosomes crosses the BBB and targets hippocampus cells of the mouse brain. FIG. 6A shows a tile scan stitched confocal micrograph representing optical Z-sections maximum intensity projected capexosomes in the hippocampus of a mouse brain. FIG. 6A shows the NeuN antibody (white) labelled hippocampal neurons.

FIG. 6B shows systemically delivered GFP-capexosomes crosses the BBB and targets hippocampus cells of the mouse brain. FIG. 6B shows a tile scan stitched confocal micrograph representing optical Z-sections maximum intensity projected capexosomes in the hippocampus of a mouse brain. FIG. 6B shows DH-labelled (red) capexosomes.

FIG. 6C shows systemically delivered GFP-capexosomes crosses the BBB and targets hippocampus cells of the mouse brain. FIG. 6C shows a tile scan stitched confocal micrograph representing optical Z-sections maximum intensity projected capexosomes in the hippocampus of a mouse brain. FIG. 6C shows a GFP (green) expression in the same hippocampal area as FIG. 6A.

In order to achieve neuron-specific targeting, the present invention provides a method for genetically engineering a joined functional AAV capsid domain specific peptide (we call it CAP) to mesenchymal stem cell derived exosomal surface via fused protein lysosome-associated membrane glycoprotein (Lamp2b)-CAP. Various steps for generating engineered exosomes with the CAP (we call capexosome) are shown in the following schematic. The functional domain designed for the invention includes endosomal escaping signals from AAV9, nuclear transport signal from AAV9, seven substituted amino acids in the variable region IV from of AAV PHP eB capsid protein, and heptamer insertion from AAV CAP-B22 followed by tetramer substitution in the variable region VIII of AAV66 for brain cell targeting without targeting liver and other off-target organs. Amino acid sequences of joined CAP and their corresponding nucleotide codons used for cloning in the N-terminal domain of peDNA Lamp2b-HA plasmid vector are presented below

```
5′-CAC GAC AAG GCC TAC GAC AGA CAG GCC AAG AAG AGG ggc agc ggc
        H D K A Y D R Q A K K R G S G
      Endosomal escaping signal Nuclear localization signal Spacer
GAC GGC ACC CTG GCC GTG CCC TTC AAG GCC ggc agc ggc ggc GTG AGC
  D G T L A V P F K A G S G G V S
  Insertion in variable region VIII of AAVS Spacer
ACC AAC CTG CAG AGC GGC AAC ACC CAG GCC GCC ACC ACC ggc agc GAC
  T N L Q S G N T Q A A T T G S D
Substitution in variable region IV of AAV9 Spacer
GAC GGC CAG AGC AGC AAG AGC-3′
  D G Q S S K S
```

Displaying specific amino acid sequence on exosomal surface for targeted brain delivery

Genetic intervention is continually explored as a therapeutic option for CNS diseases. The safety and efficacy of gene therapies relies upon expressing a therapeutic gene in affected cells while minimizing off-target expression. To achieve brain cell type specific targeting after intravenous delivery of gene, we first generated peptide sequences by combining multiple surface-exposed loops present in recently generated capsid variants that are enriched in the brain and detargeted from the liver in mice. Next, the corresponding nucleotide sequences that encode the variant loops is cloned at the N-terminus site of lamp2b expression plasmid vector that after expressing in MSC cells generated capexosomes. Thus the use of capexosome described in the present invention has potential ability to cross the BBB with brain-cell type specific in rodents as well as non-human primate and enables new avenues for basic research and potential therapeutic interventions unattainable with currently available exosome targeted brain delivery.

Advantage over brain targeted gene delivery by AAV:
1. No restriction in size of the gene for delivered
2. Reduction in the production of neutralizing antibodies due to presence of much less AAV-specific immunogenic amino acid sequences that displaying in the surface of capexosomes.
3. Much less activation of capsid-specific CDS+T cells.
4. Unlike AAV, inorganic and organic small molecule drugs can be loaded into the exosomes and delivered to the brain. Advantage over RVG-exosome mediated delivery:
5. Unlike RVG-exosomes, capexosome will deliver to the brain and not to the liver and other off-target organs.
6. Expression of AAV capsid variant specific multiple surface-exposedloops in the capexosome.
7. AAV capsid variant specific surface exposed loops used in this invention was generated by researchers from Viviana Gradinaru's lab at California Institute of Technology, Pasadena, CA, using a Cre-transgenic-based screening platform for fast and efficient capsid selection and identification of capsid variants that are enriched in brain and detargeted from the liver in mice.
8. One surface exposed loop specific heptamer substitution in variable region IV of AAV PHP eB used in the CAP peptide enable robust non-invasive gene delivery to the brain following.

This invention provides a composition for systemically delivering agents to target cells or tissues in the central nervous system of a patient comprising a brain targeted exosome derived from mesenchymal stem cells. This composition includes wherein said exosome expresses blood brain barrier (BBB) crossing central nervous system (CNS) specific AAV synthetic capsid (CAP) peptide. Certain embodiments of this invention includes this composition that is in intravenous dosage form for delivering agents, such as for example, but not limited to, inorganic and organic small molecule drugs, RNA, a gene, RNA oligos (oligosaccharides), and siRNA, specifically to the brain of said patient. Further, this composition includes wherein said exosome derived from mesenchymal stem cells are either an allogenic or an autologous mesenchymal stem cell derived exosomes. This composition crosses the blood-brain barrier.

A method is provided for genetically engineering exosomes comprising joining selected functional AAV capsid domain specific peptide (CAP), and fusing said CAP to lysosome-associated membrane glycoprotein (Lanp2b) to form CAP-Lam2b fusion protein, and expressing CAP-lamp2b fusion protein on the surface of a mesenchymal stem cell derived exosomes.

An amino acid sequence of SEQ ID NO: 1 is
provided. Amino acid sequence
SEQ ID NO: 1
HDKAYDRQAKKRGSGDGTLAVPFKAGSGGVSTNLQSGNTQAATTGSDDG
QSSKS.
A nucleotide sequence of SEQ ID NO: 2 is provided.
Nucleotide sequence
SEQ ID NO: 2
cacgacaaggcctacgacagacaggccaagaagaggggcagcggcgacg
gcaccctggccgtgcccttcaaggccggcagcggcggcgtgagcaccaa
cctgcagagcggcaacacccaggccgccaccaccggcagcgacgacggc
cagagcagcaagagc.

A composition comprising an amino acid sequence of a joined CAP cloned in a N-terminal domain of a pcDNA Lamp2b-HA plasmid vector comprising:

```
5′-CAC GAC AAG GCC TAC GAC AGA CAG GCC AAG AAG AGG ggc agc ggc
        H D K A Y D R Q A K K R G S G
      Endosomal escaping signal Nuclear localization signal Spacer
GAC GGC ACC CTG GCC GTG CCC TTC AAG GCC ggc agc ggc ggc GTG AGC
  D G T L A V P F K A G S G G V S
  Insertion in variable region VIII of AAVS Spacer
ACC AAC CTG CAG AGC GGC AAC ACC CAG GCC GCC ACC ACC ggc agc GAC
  T N L Q S G N T Q A A T T G S D
Substitution in variable region IV of AAV9 Spacer
GAC GGC CAG AGC AGC AAG AGC-3′,
  D G Q S S K S
```

and a pharmaceutically acceptable carrier, is provided.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

It is further to be understood that all values are approximate, and are provided for description.

All patents, applications, publications, test methods, literature, and other materials cited herein are incorporated by reference. If there is a discrepancy between (a) the incorporated by reference patents, applications, publications, test methods, literature, and other materials, and (b) the present application, then the present application's specification, figures, and claims control the meaning of any terms and the scope of the inventions set forth herein.

What is claimed is:

1. A composition comprising a brain targeted exosome derived from mesenchymal stem cells for systemically delivering an agent to target cells or tissues in the central nervous system of a patient.

2. The composition of claim 1 wherein said exosome expresses blood brain barrier (BBB) crossing central nervous system (CNS) specific AAV synthetic capsid (CAP) peptide.

3. The composition of claim 2 wherein said composition is in intravenous dosage form for delivering said agent that is selected from the group consisting of an inorganic molecule, an organic molecule, a drug, a RNA, a gene, a RNA oligosaccharide, and a siRNA specifically to a brain of said patient.

4. The composition of claim 1 wherein said exosome derived from mesenchymal stem cells is either an allogenic or an autologous mesenchymal stem cell derived exosomes.

5. The composition of claim 1 that crosses the blood-brain barrier.

6. A method for genetically engineering exosomes comprising joining a selected functional AAV capsid domain specific peptide (CAP), and fusing said CAP to a lysosome-associated membrane glycoprotein (Lanp2b) to form a CAP-Lam2b fusion protein, and expressing CAP-lamp2b fusion protein on a surface of a mesenchymal stem cell derived exosome.

7. An amino acid sequence of a joined CAP and corresponding nucleotide codons cloned in a N-terminal domain of a pcDNA Lamp2b-HA plasmid vector comprising:
```
5′-CAC GAC AAG GCC TAC GAC AGA CAG GCC AAG AAG AGG ggc agc ggc
        H D K A Y D R Q A K K R G S G
      Endosomal escaping signal Nuclear localization signal Spacer
GAC GGC ACC CTG GCC GTG CCC TTC AAG GCC ggc agc ggc ggc GTG AGC
  D G T L A V P F K A G S G G V S
  Insertion in variable region VIII of AAVS Spacer
ACC AAC CTG CAG AGC GGC AAC ACC CAG GCC GCC ACC ACC ggc agc GAC
  T N L Q S G N T Q A A T T G S D
Substitution in variable region IV of AAV9 Spacer
GAC GGC CAG AGC AGC AAG AGC-3′.
  D G Q S S K S
```

8. An amino acid sequence of SEQ ID NO: 1.

9. A nucleotide sequence of SEQ ID NO:2.

10. A composition comprising an amino acid sequence of a joined CAP cloned in a N-terminal domain of a pcDNA Lamp2b-HA plasmid vector comprising:
```
5′-CAC GAC AAG GCC TAC GAC AGA CAG GCC AAG AAG AGG ggc agc ggc
        H D K A Y D R Q A K K R G S G
      Endosomal escaping signal Nuclear localization signal Spacer
GAC GGC ACC CTG GCC GTG CCC TTC AAG GCC ggc agc ggc ggc GTG AGC
  D G T L A V P F K A G S G G V S
  Insertion in variable region VIII of AAVS Spacer
ACC AAC CTG CAG AGC GGC AAC ACC CAG GCC GCC ACC ACC ggc agc GAC
  T N L Q S G N T Q A A T T G S D
Substitution in variable region IV of AAV9 Spacer
GAC GGC CAG AGC AGC AAG AGC-3′,
  D G Q S S K S
```
and a pharmaceutically acceptable carrier.


SEQUENCE LISTING (2 sequences)

SEQ ID NO: 1 - protein, 54 aa, synthetic construct
HDKAYDRQAKKRGSGDGTLAVPFKAGSGGVSTNLQSGNTQAATTGSDDGQSSKS

SEQ ID NO: 2 - DNA, 162 bp, Synthetic construct
  source 1..162: mol_type = other DNA
cacgacaaggcctacgacagacaggccaagaagaggggcagcggcgacggcaccctggccgtgcccttcaaggccggcagcggcggcgtgagcaccaacctgcagagcggcaacacccaggccgccaccaccggcagcgacgacggccagagcagcaagagc